(19) United States
(12) Patent Application Publication
(10) Pub. No.: US 2004/0219614 A1
(43) Pub. Date: Nov. 4, 2004

(54) Monoclonal antibody specific to truncated midkine (tMK) protein and uses thereof

(76) Inventors: Tomohiro Mitsumoto, Mitsuke-shi (JP); Takao Shinozawa, Kumagaya-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/427,961

(22) Filed: May 2, 2003

(51) Int. Cl. G01N 33/574; C07K 16/24

(52) U.S. Cl. 435/7.23; 530/388.23

(57) ABSTRACT

The present invention relates to a monoclonal antibody or a fragment thereof specific to truncated Midkine (tMK) protein, hybridoma producing the monoclonal antibody, a method of detecting truncated Midkine protein (tMK) by use of the monoclonal antibody, a method of detecting a tumor cell, and a kit containing the monoclonal antibody for detecting truncated Midkine (tMK) protein.

### BACKGROUND OF THE INVENTION

1. Field of the Invention

The present invention relates to a monoclonal antibody specific to truncated Midkine (tMK) protein or a fragment thereof, a hybridoma producing the monoclonal antibody, a method of detecting the truncated Midkine (tMK) protein by use of the monoclonal antibody, a method of detecting tumor cells by use of the monoclonal antibody, and a detection kit containing the monoclonal antibody for the truncated Midkine (tMK) protein.

2. Description of the Related Art

“Midkine (MK)” is a growth factor which was found as a gene product responsive to a retinoic acid during the differentiation of embryonal tumor cells. MK has been reported to have a heparin binding ability and plays roles in growth and differentiation of nerve cells, neovascularization, and plasminogen-activity enhancement in the endothelial cells. Through these actions, MK is assumed to concern with the carcinogenesis. According to the report of K. Kadomatsu et al. [Br. J. Cancer, 75 354-359 (1997)], it was confirmed that MK expression increases in Wilms' tumor, stomach cancer, colon cancer, and others compared to the normal tissues, and that if MK is overexpressed in mouse fibrobrast cells NIH3T3 by introducing an Mk gene, the fibrobrast cells become cancerous. MK is a protein rich in basic amino acids having a molecular weight of 13,000 [M. Tomomura et al., J. Biol. Chem., 265, 10765-10770 (1990)] and composed of two domains, N-domain (1 to 61 amino acids) and C-domain (62 to 121 amino acids)[L. Fabri et al., J. Chromatogr., 213-225 (1993)]. The active site of MK is localized in the C-domain [H. Muramatsu et al., Biochem. Biophys. Res. Commn., 203, 1131-1139 (1994)].

On the other hand, it was found, in 1996, that short-form MKmRNA (280 bp) is expressed in tumor cells by PCR using a MK primer for full-length MKmRNA [I. Miyashiro et al., Cancer Letters 106, 287-291 (1996); T. Kaname et al., Biochemical and Biophysical Research Communications, 219, 256-260 (1996)]. The short-form MKmRNA is called “truncated Midkine mRNA”(tMKmRNA), which is a mutant of full-length MKmRNA, lacking the third exon from the five exons. The protein structure of tMKmRNA estimated from the sequence of the mRNA lacks the N domain of MK and thus composed of a part of the N-terminal and the C-domain serving as a main active site. However, the presence of tMK protein has not yet been confirmed and a method for detecting the tMK protein has not been established.

Examples of the tumor cells in which the expression of tMKmRNA has been hitherto confirmed, include Wilms' tumor, pancreas cancer, stomach cancer, lung cancer, and colon epithelial cancer. However, tMKmRNA is not expressed in the normal cells (non-tumor cells) of the aforementioned organs [see, for example, K. Aridome et al., British Journal of Cancer, 78, 472-477 (1988)]. It is also reported that the expression of tMK may be used as a diagnostic marker for metastasis of cancer from the stomach to the lymph node [see K. Aridome et al., British Journal of Cancer, 78 (4), 472-477 (1988)].

Detection and characterization of tumor is the most important role of the diagnosis and treatment of cancer. Currently, a tumor is generally diagnosed by microscopic observation of cells or tissue pieces. However, such microscopic morphological observation called cytodiagnosis has several problems. For example, in some cases, a specimen for examination is not taken sufficiently, making diagnosis difficult. In other cases, a specimen itself cannot be taken. For these reasons, no less than 50% of tumor patients cannot be diagnosed. In most cases, no clear evidence and proof of cancer are not given. In these cases, since the sufficient amount of detached cells is not taken as specimen, needling must be performed, which gives a large burden to the patient. In addition, specific skills and experience are required since the symptom of cancer is difficult to be generalized. It follows that a large number of specimens cannot be determined quickly.

On the other hand, the prognosis is also made depending upon morphological observation under microscopy. In general, as the degree of morphological irregularity of the primary tumor cells increases, the possibility of metastasis increases. However the correlation between them has not been elucidated. To choose the best treatment, it is useful to know the possibility of metastasis accurately.

Recently, monoclonal antibodies having excellent specificities have been successfully produced. As a result, cancer diagnosis has made remarkable progress. In the monoclonal-antibody diagnosis, selection of a tumor marker, that is, a detection target, is critical. The tumor marker is a substance produced from tumor cells, such as a substance produced exclusively in tumor cells, a substance which may be produced in non-tumor cells but enormously produced particularly in tumor cells, or a substance produced from non-tumor cells as a result of a normal biological reaction to malignant proliferation. Examples of well-known tumor markers include α-fetoprotein (AFP) and cancer embryonic antigen, which are used for monitoring progress of cancer and effects of the treatment. Unfortunately, such tumor markers have problems. As a matter of fact, some tumor markers are detected in non-tumor cells and other tumor markers are not detected until tumor tissue grow up to certain sizes. Still other tumor markers are detected only in a specific tumor. In contrast, unlike these tumor markers, tMK expresses in a wide variety of tumor cells and never expresses in non-tumor cells, as described above. Therefore, if a specific detection method for tMK is developed, tMK will be an excellent marker for various tumor cells in a diagnosis.

### BRIEF SUMMARY OF THE INVENTION

According to the present invention, the following seven aspects (1) to (7) are provided.

(1) A monoclonal antibody or a fragment thereof capable of reacting with truncated Midkine (tMK) protein and incapable of reacting with Midkine (MK) protein;

(2) A monoclonal antibody or a fragment thereof according to Section (1) above, in which the truncated Midkine (tMK) protein has an amino acid sequence composed of the full-length amino acid sequence of the Midkine protein minus the sequence encoded in the third exon;

(3) A hybridoma producing a monoclonal antibody according to Section (1) above and prepared by fusing a mouse spleen cell immunized with the truncated Midkine protein and a mouse myeloma cell;

(4) A method of detecting truncated Midkine (tMK) protein expressed specifically in a tumor cell by use of a monoclonal antibody or a fragment thereof according to Section (1) above;

(5) A method of detecting a tumor cell by detecting truncated Midkine (tMK) protein expressed specifically in a tumor cell by using a monoclonal antibody or a fragment thereof according to Section (1);

(6) A kit for detecting truncated Midkine protein in which the kit comprises a monoclonal antibody or a fragment thereof according to Section (1); and

(7) Truncated Midkine (tMK) protein and a homologous substance recognized specifically by a monoclonal antibody or a fragment thereof according to Section (1).

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

FIG. 1 shows amino acid sequences of human Midkine protein and recombinant truncated Midkine protein;

FIG. 2 shows the Western blotting results of tMK in the supernatant of G401 cell culture. Lane 1 shows purified recombinant tMK protein (2.5 μg/lane) and lane 2 shows partially purified tMK protein (8 μg/lane) contained in the supernatant of the G401 cell culture. The relative molecular mass (kDA)of a standard protein is shown in the left-hand side of the figure;

FIG. 3 shows G401 cells immunologically stained with anti-tMK-MiStMK-V3 antibody;

FIG. 4 shows a section of human Wilms' tumor tissue immunologically stained with anti-tMK-MiStMK-V3 antibody;

FIG. 5 shows the results of ELISA analysis for binding an anti-tMK-scFV fragment to tMK;

FIG. 6 shows the results of ELISA analysis for binding an anti-tMK-scFV fragment to full-length MK, MK c-half (MK sequence of amino acids 62-121) and a recombinant tMK; and

FIG. 7 shows inhibition of the binding of an anti-tMK-MiStMK-V3 antibody to MK by an anti-tMK-scFV fragment.

### DETAILED DESCRIPTION OF THE INVENTION

The present inventors have conducted intensive studies and succeeded in fusing a mouse spleen cell immunized with truncated Midkine (tMK) protein and a mouse myeloma cell to produce a hybridoma, and obtaining a monoclonal antibody capable of specifically recognizing the truncated Midkine (tMK) protein, from the hybridoma. Based on the achievement, the present invention was attained.

### 1. Production of Recombinant Truncated Midkine Protein

Recombinant truncated Midkine protein can be obtained by expressing a human MK gene fragment lacking the third exon in Escherichia coli (E. coli) and purifying the expression product.

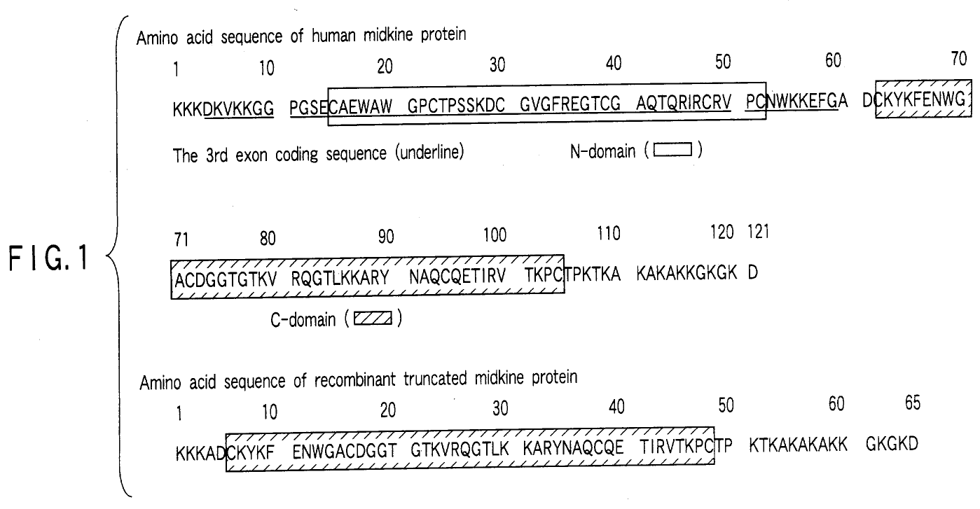

The term “Midkine protein” used in the invention refers to a protein composed of a full-length amino acid sequence having 121 amino acids, as shown in FIG. 1. The term “truncated Midkine protein” refers to a protein having a 65 amino-acid sequence composed of the full-length amino acid sequence of the MK gene minus that encoded in the third exon. Hereinafter, the “Midkine protein” will be represented by “MK” and the “truncated Midkine protein” by “tMK”.

### 2. Production of tMK Protein Specific Monoclonal Antibody

A tMK protein specific monoclonal antibody according to an aspect of the present invention is, for example, produced as follows.

(1) Immunity Animals and Sampling of Antibody-Producing Animal Cells

The recombinant tMK protein obtained in Section 1 above is administered as an antigen (immunogen) to a 3-10 week-old mouse, preferably, a 4 week-old mouse. Any immunization method may be employed as long as it is conventionally used. The antigen is preferably injected, intravenously, subcutaneously, and intraperitoneally, together with an appropriate adjuvant, such as a commercially available Freund's complete adjuvant Freund's incomplete adjuvant; BCG; aluminium hydroxide gel; and/or pertussis vaccine; and the like. Although the interval and times of the immunization are not particularly limited, immunization may be performed every one to two weeks and two to five times. The amount of antigen may be 10 to 500 μg/mouse per time.

After the 3 to 10th days from the final immunization, antibody-producing cells are collected. Examples of the antibody-producing cells include the spleen cells, lymph node cells, thymus cells, and peripheral-blood cells. Of them, the spleen cells are generally used. Such antibody-producing cells are desirably prepared by taking out the spleen, lymph node, and thymus, etc., or collecting the peripheral blood, etc., mincing the collected samples, suspending the minced pieces in medium or buffer such as PBS, DMEM, PRMI1640 or E-RDF, filtrating the suspension through a 200-250 μm stainless mesh, and subjecting centrifugal separation.

(2) Cell Fusion

As the myeloma cells to be fused with the antibody-producing cells, a commercially available mouse cell strain may be used. The cell strain to be used herein preferably has drug resistance and cannot live in a non-fused state but can live only in a fused state with antibody-producing cells in a selective medium (e.g. HAT medium). Generally, 8-azaguanine-resistant cell strain is used. Since this cell strain lacks hypoxanthine-guanine phosphoribosyltransferase (HGPRT), it cannot grow in hypoxanthine aminopterin thymidine (HAT) medium. Specific examples of the myeloma cells include mouse myeloma cell strains such as Sp2/0-Agl4 [ATCC CRL-1581; Nature, 276, 270-272 (1978)], P3X63Ag8[ATCC TIB-9; Nature, 256, 495-497 (1978)], P3X63 Ag8U.1 (P3U1)[AtCC CRL-1580; Current Topics in Microbiology and Immunology, 81, 1-7(1978); Antibodies A LABORATORY MANUAL., Ed Harlow., David Lane., Cold Spring Harbor Laboratory, (1988)], P3X63Ag8.653 [ATCC TIB-18; European J. Immunology, 6, 511-519 (1976); Antibodies A LABORATORY MANUAL., Ed Harlow., David Lane., Cold Spring Harbor Laboratory, (1988)], and P2/NSI/1-Ag4-l[ATCC CRL-1581; Nature, 276, 269-270 (1978)].

The antibody-producing cells immunized in the section (1) above are fused with the myeloma cells obtained above. Cell fusion is efficiently performed by bringing the myeloma cells of 10⁷ to 10⁸ cells/mL into contact with the antibody-producing cells in a mixing ratio from 1:1 to 1:10, for example, about 1:5, in animal-cell culture medium such as MEM, DMEM, PRMI-1640 or E-RDF in the presence of a fusion-accelerating agent at 30 to 37° C. for 1 to 3 minutes. To accelerate the cell fusion, a fuse-accelerating agent such as polyvinyl alcohol or polyethylene glycol having an average molecular weight of 1000 to 6000, or fusion virus such as Sendai virus may be used. In addition, the antibody-producing cells and the myeloma cells may be fused in the presence of electric stimulus (e.g., electroporation) applied by a commercially available cell-fusion apparatus.

(3) Screening and Cloning of Hybridoma

Desired hybridoma is screened from the cells after cell-fusion process. The screening is performed by selective proliferation performed in selective medium. More specifically, a cell suspending solution is added to, for example, Iscove's medium (IMDM) supplied with a HAT supplement (Gibco BRL) and interleukin-6 (1 unit/ml) and diluted with the medium (IMDM) so as to obtain a concentration of 10³ to 10⁷ cells/mL. Thereafter, the diluted cell suspension is added to 96 wells of a cell-culture microplate in a concentration of 10² to 10⁶ cells per well and subsequently a selective medium such as HAT medium is added. Culture is performed while replacing the HAT medium with a fresh one at appropriate intervals.

When 8-azaguanine resistant cell strain is used as the myeloma cells and HAT medium as a selective medium, non-fused myeloma cells die off by about the 7th to 10th day after the initiation of culturing, and the antibody-producing cells, even though they are non-tumor cells, cannot survive for a long time in vitro and also die off by the 7th to 10th day of the culturing. As a result, hybridoma cells, which start growing before or after the 6th to 10th day of culturing, can be obtained.

Subsequently, the supernatant of the cell culture containing amplified hybridoma cells is screened as to whether or not it contains the desired tMK antibody. The screening method for the hybridoma cells is not particularly limited and a general screening method may be used. More specifically, an aliquot is taken from the supernatant of the culture containing the hybridoma cells in a well and added to another well having tMK antigen fixed thereto. Thereafter, a labeled secondary antibody is added to the well and subjected to incubation. The binding ability is checked by Enzyme Linked Immunosorbent Assay (ELISA) or radioimmunoassay (RIA).

To explain more specifically, the supernatant of a culture containing monoclonal antibody was added to the 96 wells of a microplate having tMK antigen as an immunogen fixed thereto to allow the monoclonal antibody to bind to the tMK antigen. Next, the monoclonal antibody bound with the antigen is allowed to react with an enzyme-linked anti-immunoglobulin antibody. Subsequently, an enzyme substrate is added to each well to produce a color. Since the color is produced only in the tMK-antigen fixed well as an immunogen and containing the desired monoclonal antidody, a colored supernatant is selected. In this manner, the desired hybridoma producing the antibody capable of binding to the tMK antigen can be screened. The cloning of the hybridoma is performed by the limiting dilution analysis, soft agar culture, fibringel method, or by means of a fluorescence activated cell sorter. Finally, the monoclonal-antibody producing hybridoma can be obtained.

(4) Collection of Monoclonal Antibody

The monoclonal antibody may be collected from hybridoma obtained above by the cell culture method or the ascites formation method generally used. For example, in the cell culture method, the hybridoma cells are cultured in an animal-cell culture medium, such as IMDM, RPMI-1640, MEM or E-RDF containing a 10 to 20% fetal bovine serum, or a serum-free culture medium for 2 to 14 days under general culturing conditions (e.g., 37° C., 5% CO₂) and the monoclonal antibody is obtained from the culture supernatant.

In the ascite formation method, a mineral oil such as pristane (2,6,10,14-tetramethylpentadecane) is injected into the peritoneal cavity of the same mammalian species as that from which the myeloma cell is derived. Thereafter, hybridoma cells of 1×10⁷ to 1×10⁹, and more preferably, 5×10⁷ to 1×10⁸, are injected into the peritoneal cavity to proliferate the hybridoma cells in a large amount. The ascite or the serum is collected after 1 to 4 weeks and preferably after 2 to 3 weeks.

In the monoclonal antibody collection method, the antibody may be purified by a known method or combination of known methods. Example of the known methods include an ammonium sulfate salting out method, ion-exchange chromatography using an anion exchanger such as DEAE cellulose, affinity chromatography using protein A Sepharose, etc., and molecular sieve chromatography which separates a substance depending upon a molecular weight or configuration. Through the purification process, the tMK specific monoclonal antibody can be obtained.

### 3. Detection of tMK by the Monoclonal Antibody of the Invention

The detection of tMK by the tMK specific monoclonal antibody according to an aspect of the invention may be performed by, for example, immuno-blotting, enzyme immunoassay (EIA), radioimmunoassay (RIA), fluorescent antibody technique, or the immunostaining method, however not limited thereto. As example of the specimen used herein include pieces of the tumorigenic suspect tissue, blood, lymph, sputum, pulmonary toilet liquid, urine, fetus, and tissue culture supernatant but not limited to these.

As the tMK specific monoclonal antibody, a fragment thereof, more specifically, a single chain antibody fragment (scFv) of the tMK specific monoclonal antibody may be used. For example, the tMK specific monoclonal antibody is detected by the ELISA as follows. First, a specimen such as diluted blood is fixed to a 96-well microplate and the tMK specific monoclonal antibody serving as a primary antibody is reacted with the specimen. Subsequently, an anti-globulin antibody labeled with a specific enzyme such as POD (peroxidaze) required for a color-producing reaction is reacted with the monoclonal antibody. Thereafter, the reaction solution is washed and ABTS (′12,21′-azino-di-(3-ethyl-benzothiazoline-6-sulfonic acid) or the like is added to the reaction solution, as a color-producing substance. The presence of tMK in the specimen is calorimetrically detected.

The tMK specific monoclonal antibody is also detected by sandwich ELISA as follows. First, a diluted specimen such as blood is added to a 96 microplate to which the tMK specific monoclonal antibody has been absorbed in advance. The microplate is incubated for a predetermined time and washed. Thereafter, a purified antibody labeled with biotin is added to each well and incubated for a predetermined time. After the incubation, the plate is washed and enzyme-labeled avidin is added to the wells. After the microplate is further incubated, the resultant plate is washed and orthophenylenediamine is added as a color-producing substrate to the wells. Produced color is calorimetrically detected.

### 4. TMK Detection Kit Containing the Monoclonal Antibody of the Invention

The tMK detection kit according to an aspect of the present invention may not be particularly limited as long as it contains at least the tMK specific monoclonal antibody of the present invention. A preferable tMK detection kit includes a monoclonal antibody to be fixed to a solid phase and another monoclonal antibody serving as a secondary antibody. Both monoclonal antibodies differ in recognition site. The monoclonal antibody serving as a secondary antibody may be labeled with a marker substance such as an enzyme. Other than the two monoclonal antibodies, the tMK detection kit may include various agents such as an enzyme substrate, buffer and/or dilution liquid, etc.

As mentioned above, the tMK specific monoclonal antibody of the present invention makes it possible to accurately detect tMK in biological specimens such as cells and tissues. The tMK specific monoclonal antibody can be used in tumor diagnosis, screening for a risk group, prediction of cancer metastasis, and monitoring the progress of cancer. Furthermore, administration of the tMK specific monoclonal antibody makes it possible to inhibit tumor formation. If the tumor growth inhibitor is attached to the monoclonal antibody and administered, tumor cells can be selectively eliminated. The monoclonal antibody of the present invention is therefore useful in treating and preventing tumors.

It has been recently revealed that the truncated midkine can be synthesized also in the liver during the development of a human being (this finding will be reported in M. Kato, T. Shinozawa, S. Kato and T. Terada; Histology and Histopathology 2003;18:129-134). By this finding, the usefulness and importance of the present invention are strongly supported.

### EXAMPLES

Now, the present invention will be described in detail below by way of examples, which should not be construed as limiting the scope of the present invention.

### Example 1

### Preparation of Recombinant tMK Protein

On the basis of information of tMKmRNA [T. Kaname et al., Biochemical and Biophysical Research Communications vol. 219, pp. 256-260 (1996)], four amino acids, Met-Lys-Lys-Lys, were additionally introduced into the N-terminal of the amino acid sequence (60-121 amino acids) of a human MK fragment to construct a tMK expression plasmid. First, a plasmid vector (pUC118-MK vector) containing the MK sequence was subjected to PCR using a sense primer: 5′-GCC CAT GGG GATGAA AAA GAA AGC CGA CTG-3′ (Sequence No. 1) where the portion of “CC CAT GG” is an NcoI restriction-enzyme recognition site, an antisense primer: 5′-CCC AAG CTT AGT CCT TTC CCT TCC CTT TCT-3′ (Sequence No. 2) where a portion of “AAG CTT” is a Hind III restriction enzyme recognition site. In this manner, a DNA fragment encoding tMK protein was obtained. The PCR cycle (94° C. for one minute, 55° C. for one minute and 72 ° C. for 30 minutes) was repeated 30 times. The sequence of a PCR product (217 bp) is determined by an automatic DNA sequence analyzer (DSQ-1000, Shimazu, Japan) using a TA sequence vector (Novagen, USA). The DNA sequence of the PCR product is shown under Sequence No. 3 and the corresponding amino acid sequence under Sequence No. 4.

The plasmid containing a tMK gene was digested with NcoI and HindIII. The digested product with the restriction enzymes was loaded to agarose-gel electrophoresis. The obtained DNA fragments were purified by Gene Clean kit (Bio 101, Inc., USA). Each of the purified DNA fragment was ligated with a pelB leader sequence present downstream of T7 promoter within an expression vector, pET-25b (+), by T4 DNA ligase (Ligation Kit, Takara, Japan) to construct pET-25b (+)-tMK plasmid. After E. coli BL21 was transfected with the pET-25b(+)-tMK plasmid, positive clones were obtained in an LB agar plate containing ampicilline (100 μg/mL).

E. coli BL21 harboring the pET-25b(+)-tMK plasmid, was cultured in 2 × YT medium containing ampicilline (100 μg/mL) and 0.1 mM IPTG (Isopropyl-1-thio-D-galactopyranoside). The cultured cells were centrifugally collected and sonically crushed. The precipitation obtained by the centrifugal separation was suspended in soluble buffer [20 mM Tris-HCl (pH7.6), 8.0 M urea, 10 mM DTT, and 0.1 mM PMSF (phenylmethanesulfonyl fluoride), placed at room temperature for 6 hours, and subjected centrifugal separation to obtain a supernatant. The supernatant was dialyzed against a buffer [20 mM Tris-HCl (pH8.5), 0.1M NaCl, 0.1 mM PMSF, 1.0 mM CaCl₂, and 1.0 mM MgCl₂]. The resultant mixture was subjected to another centrifugal separation and the supernatant was dialyzed against buffer A [50 mM Sodium phosphate buffer pH6.8, 0.1 mM PMSF]. The dialyzed solution was loaded to a Hi-Trap Heparin column (volume 5 mL; Pharmacia Biotech, Uppsala) and eluted with buffer A containing 1.5 M NaCl. The purity of recombinant tMK protein thus obtained was determined by SDS-PAGE with CBB staining and Western blotting using anti-MK antibody [S. Kato et al., J. Neuropath and Exp. Neurogy, 58, 430-441 (1999)].

### Example 2

### Preparation of Anti-tMK Specific Antibody

(1) Preparation of Anti-tMK Specific Antibody Producing Cell

First, 7 week-old BALB/C line female mouse was immunized by administering the recombinant tMK protein prepared in Example 1 as an antigen every two weeks, three times in total. The first and second immunization, Freund's complete adjuvant (FCA) and Freund's incomplete adjuvant (FICA) were mixed in an equal amount, emulsified, and subcutaneously administered to the mouse. In the third immunization, the same amount of antigen as those of the first and second immunization was injected through the tail vein. At the third day after the final immunization, the spleen cells were fused with mouse myeloma cells P3×63-AG8.653 in a ratio of 5:1 by use of PEG and cultured in HAT selective medium to select hybridoma cells alone. The obtained hybridoma cells were seeded to 96 wells of a microplate (0.5 cell/well) in accordance with the limiting dilution analysis. Thereafter, the hybridoma cells forming a single colony in the wells were used as a clone. Such limiting dilution analysis was repeated twice to perform cloning. The culture supernatant of the cloned hybridoma was subjected to the anti-tMK specific antibody detection described below to establish hybridoma producing the anti-tMK specific antibody.

(2) Detection of Anti-tMK Specific Antibody

The tMK protein solution (antigen solution) prepared in Example 1 was dispensed in the 96 wells of a microplate by 100 ng/well and allowed to stand overnight at 4° C. or room temperature for 2 hours to fix the protein to the well. As a control, an MK protein solution was added and fixed to the 96 wells of another microplate in the same manner. After the antigen solution (or MK protein solution) was removed, blocking was performed by a 1% BSA/PBS solution. In this way, an anti-tMK specific antibody detection plate and a control plate were prepared. The hybridoma culture supernatant prepared in the section (1) was added dropwise to each well of both plates and incubated at room temperature for 60 minutes. After the incubation, the hybridoma culture supernatant was discarded and the remaining hybridoma culture supernatant in the well was washed out with PBS. Subsequently, a POD-labeled anti-mouse IgG antibody solution (1:200 dilution, manufactured Wako Pure Chemical Industries Ltd.) was added dropwise by 50 μL/well. The resultant microplates were incubated at room temperature for 60 minutes. After the POD labeled anti-mouse IgG antibody solution was discarded, the wells were washed with PBS. Subsequently, a commercially available ABTS (2,2′-azino-di-(3-ethyl-benzothiazoline-6-sulfonic acid) solution (as a POD substrate solution) was added dropwise by 100 μl/well and incubated at room temperature for 10 to 20 minutes. Thereafter, the absorbancy of there resultant solutions at 415 nm was determined by a microplate reader. The results are shown in Table 1.

TABLE 1

OD 415 nm

| | | Anti-MK activity |
|---|---|---|
| Clone | Anti-tMK activity | (control) |
| MiStMK-V1 | 0.906 | 0.187 |
| MiStMK-V3 | 1.406 | 0.186 |
| MiS-N | 1.660 | 1.696 |
| MiS-O | 1.683 | 1.48 |
| MiS-D | 1.866 | 0.794 |

The hybridoma culture supernatant whose absorbancy in the tMK protein well was 0.3 or more and absorbancy in the MK protein well (control) was 0.3 or less, was defined as positive. As a result, two positive clones were obtained. The anti tMK specific antibody produced by the hybridoma of clone No. MiStMK-V3 was designated as a “anti-tMK-MiStMk-V3 antibody”. Note that hybridoma cell strain MiStMK-V3 producing the anti-tMK-MiStMK-V3 antibody has been deposited at the National Institute of Bioscience and Human-Technology, Agency of Industrial Science and Technology under accession number FERM P-18069, as of October 3, 2000.

### Example 3

### Detection of tMK in Tumor Cells by Western Blotting

(1) Preparation of G401 Cell Supernatant

The Wilms' tumor derived G401 cells were cultured in a 90-mm petri dish containing McCoy's 5A medium supplemented with a 10% FBS up to confluent. The culture supernatant was purified by a Haparin-Sepharose column.

(2) Detection of tMK from G401 Cell Culture Supernatant By ELISA

The supernatant obtained in Section (1) was fixed to a 96-well plate for ELISA. Anti-tMK-MiStMK-V3 antibody was dispensed to individual wells as a primary antibody. As a control, anti-Mk antibody was used. These antibodies were incubated and washed and subsequently POD labeled anti-mouse IgG and POD labeled anti-rat IgG serving as a secondary antibody were dispensed and incubated. After washing, ABTS was added as a color-producing substrate and the absorbancy at 415 nm was determined. The results are shown in Table 2. As is apparent from Table 2, the presence of tMK was determined in the G401 cell culture supernatant.

TABLE 2

| Purified tMK | | | | |
|---|---|---|---|---|
| concentration | | | | |
| (ng/well) | 0 | 10 | 20 | 50 |
| Anti-tMK antibody | 0.05 | 0.16 | 0.23 | 0.58 |
| Anti-MK antibody | 0.04 | 0.05 | 0.05 | 0.06 |

(3) Detection of tMK in G401 cell culture supernatant by Western Blotting

The culture supernatant obtained in Section (1) was subjected to Tricin SDS-PAGE in accordance with the method of Schagger et al. After the electrophoresis, the separation results were transferred to a PVDF film in accordance with the method of Towbin et al. As a control, the tMK protein produced in Example 1 was subjected to the same treatment.

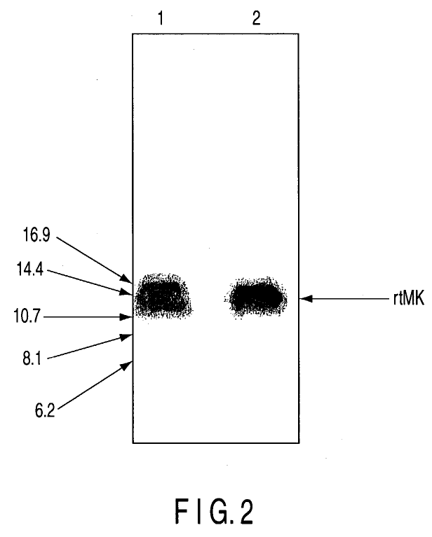

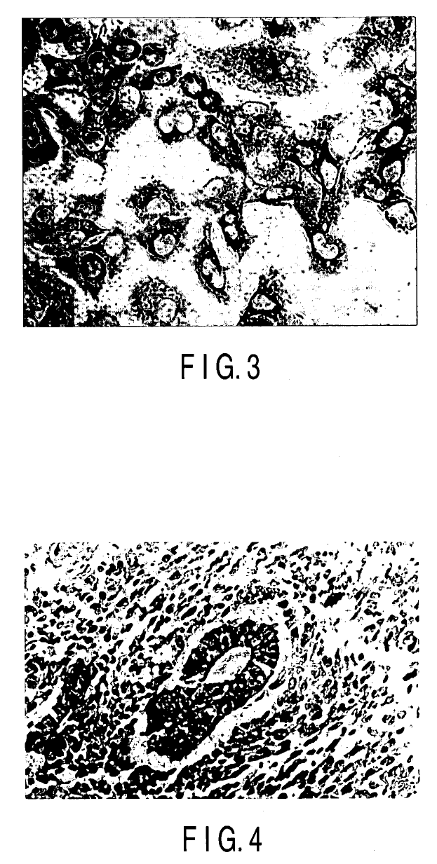

The PVDF film after the transfer was blocked with a 1% skim milk PBS and incubated using anti-tMK-MiStMk-V3 antibody as a primary antibody. Subsequently, the PVDF film was washed and incubated by using POD-labeled anti-mouse IgG as a secondary antibody. After washing, 4 chloronaphtol was added as a color-producing substrate and the PVDF film was incubated. Band was detected and carefully observed. As shown in FIG. 2, a single band at a molecular weight of about 8,000 was observed both in the lane of G401 cell culture supernatant and in the lane of the tMK antigen after the electrophoresis.

### Example 4

### Observation of tMK in Tumor Cells by Immunostaining

(1) Preparation of Fixed Sample

G401 cells were cultured on a cover glass and fixed with a periodate lysine paraformaldehyde (PLP) solution.

(2) Immunostaining

A 3% goat normal serum was added dropwise on the cover glass prepared in Section (1) and allowed to stand alone at room temperature for 30 minutes. Thereafter, anti-tMK-MiStMk-V3 antibody was added dropwise on the cover glass and allowed to stand alone at room temperature for one hour. After the cover glass was washed well in 0.1% BSA/PBS solution, 50 μL/glass of biotin-added anti-mouse IgG was added dropwise and allowed to stand at room temperature for 30 minutes. The cover glass was washed in 0.1% BSA/PBS solution and thereafter 1 mL of MeOH+8.6 μL H₂O₂ solution was added dropwise to the cover glass in a concentration of 50 μL/glass. After the cover glass was washed in the same manner as above, A solution and B solution attached to an ABC kit were added dropwise independently in 50 μL/glass and allowed to stand at room temperature for 30 minutes. After being washed well with PBS(−), 100 μL/glass of DBS was added as a substrate to produce a color. After confirming sufficient color production, the cover glass was washed with a large volume of distilled water. Furthermore, the cover glass was dehydrated with 50-100% EtOH, EtOH/xylene (1:1) solution, and 100% xylene, successively. The resultant cover glass was sealed with Canada balsam on a slide glass. As a result, an intensively stained portion was observed in the G401 cytoplasm. This suggested that tMK was expressed in the G401 cytoplasm.

(3) Staining of human tissue section

The tissue section of human Wilms' tumor was fixed with 10% phosphate buffered formalin (pH 7.6) and stained in the same manner as mentioned in Section (2) above. As a control, hematoxine/eosin staining was performed. As a result, the cytoplasma of the tumor cells differentiated into tubular form and the cytoplasma of the blast cells were stained to exhibit intensive color, as shown in FIG. 4. However, non-tumor cells were not stained.

### Example 5

### Preparation of Antibody Fragment and Detection of tMK

(1) Preparation of Anti-tMK Antibody (Anti-tMK-scFV Fragment) by Gene Recombination Technique

First, mRNA was separated from hybridoma strain MiStMk-V3 producing anti-tMK-MiStMk-V3 antibody by a commercially available kit and a cDNA library was constructed. Subsequently, PCR was performed by using a VH primer (5′-CGG AAT TCG GTG CAG CTG CAG CAG TCT GG-′) (for a 5′ terminal: Sequence No. 5), 5′-CGGCTC GAG TGA GGA GAC GGT GAC TGA GG-3′ (for a 3′ terminal, Sequence No. 6), and a VL primer 5′ GCG GAT CCT GAT GTT TTG ATG ACC CAA-3′ (for a 5′ terminal, Sequence No. 7), 5′-CCC AAG CTT TTC CAA TTT GGT GCC CGC TCC GG -3′ (for a 3′ terminal, Sequence No. 8). As a result, VH and VL regions were reproduced. The VL and VH were bound by interposing Liner(GGC GGC GGT GGC TCG) between them to form a construct of VL-liner-VH. The construct was inserted into expression vector pET-22b(+). E. coli BL21 was transformed by the vector. The transformed E. coli was cultured in 2×YT medium containing 0.1 mM IPTG to allow the anti tMK-scFV fragment to express. The expressed product was purified by a nickel chelate column.

(2) Reaction of Recombinant tMK by ELISA

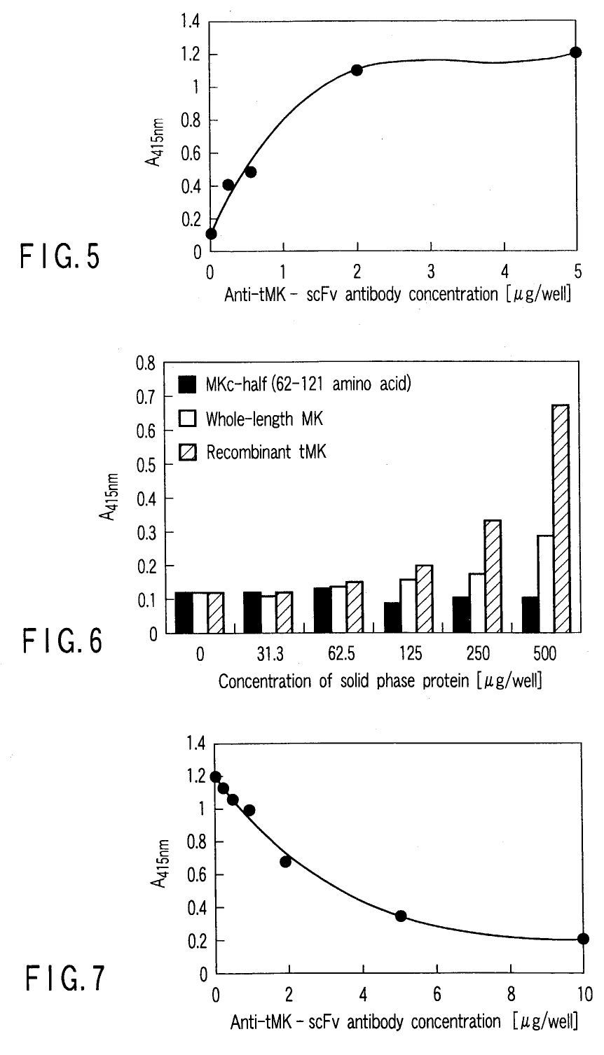

First, tMK (200 ng/100 μg) was fixed on an ELISA plate, followed by blocking it. Thereafter, the anti tMK-scFV fragment was added as a primary antibody in concentrations of 0, 0.25, 0.5, 2, 5 μg/well and incubated at room temperature for 2 hours. After the reaction, the ELISA plate was washed, anti-His-Tag (mouse IgG) was added as a secondary antibody, and incubated at room temperature for 1.5 hours. After washing, a tertiary antibody, POD-labeled anti mouse IgG antibody, was added and incubated at room temperature for one hour. After the reaction, the plate was washed, and ABTS was added as an enzyme substrate. The reaction mixture was incubated at room temperature for 15 minutes. Thereafter, the absorbancy of each well at 415 nm was measured. The results are shown in FIG. 5. As shown in FIG. 5, the more increased concentration of anti-tMK-scFV fragments are added, the higher the absorbancy. Hence, it is proved that the anti-tMK-scFV fragment is bound to tMK.

(3) Reaction with the Whole or Part of MK Protein

Full-length MK (fMK), MKc-half (MK amino acid sequence 61-121), or recombinant tMK were fixed to an ELISA plate in concentrations of 0, 31.25, 62.5, 125, 250, 500 μg/well. To the ELISA plate, an anti-tMK-scFV fragment as a primary antibody and the same secondary and tertiary antibodies as mentioned in Section (2) were added and subjected to the same ELIZA as in Section (2). The results are shown in FIG. 6. As shown in FIG. 6, the reactivity between the anti-tMK scFV fragment and the recombinant tMK is high. The anti-tMK sxFV fragment reacts slightly with the full-length MK and does not react with MKc-half. From this, it was suggested that the anti-tMK-scFV fragment is a protein recognizing a tMK specific sequence.

(4) Antagonistic Reaction between anti-tMK-MiStMK-V3 and Anti-tMK-scFV Fragment.

Recombinant tMK (200 ng/μL) was fixed on an ELISA plate. Both anti-tMK-MiStMK-V3 antibody (1 μg/well) and the anti-tMK-scFV fragment (the concentration is shown in FIG. 7) were added to each well and incubated at room temperature for 2 hours. After the reaction, the plate was washed and POD-labeled anti-mouse IgG antibody was added as a secondary antibody and incubated at room temperature for one hour. After the reaction, the plate was washed and ABTS was added as an enzyme substrate and then incubated at room temperature for 15 minutes. Thereafter, the absorbancy was measured at 415 nm. The results are shown in FIG. 7. As shown in FIG. 7, the absorbancy decreases along with decrease of the addition amount of anti-tMK-scFV fragment. Since the anti-tMK-scFV fragment does not react with the POD-labeled anti-mouse IgG, the absorbency is proportional to the amount of the anti-tMK-MiStMK-V3 antibody. Therefore, it was suggested that the anti-tMK-MiStMK-V3 antibody and the anti-tMK-scFV fragment are antagonally bound to tMK, in other words, they bind to the same site of the tMK.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A monoclonal antibody or a fragment thereof which reacts with truncated Midkine protein and which does not react with Midkine protein.

2. A monoclonal antibody or a fragment thereof according to claim 1, wherein the truncated Midkine protein has an amino acid sequence composed of the full-length amino acid sequence of the Midkine protein minus the sequence encoded in the third exon.

3. A monoclonal antibody or a fragment thereof according to claim 1, wherein the monoclonal antibody is produced from a hybridoma prepared by fusing a mouse spleen cell immunized with the truncated Midkine protein and a mouse myeloma cell.

4. A hybridoma producing a monoclonal antibody according to claim 1 and prepared by fusing a mouse spleen cell immunized with the truncated Midkine protein and a mouse myeloma cell.

5. A method of detecting truncated Midkine protein expressed specifically in a tumor cell by use of a monoclonal antibody or a fragment thereof according to claim 1.

6. A method of detecting a tumor cell by detecting truncated Midkine protein expressed specifically in a tumor cell by using a monoclonal antibody or a fragment thereof according to claim 1.

7. A kit for detecting truncated Midkine protein, comprising a monoclonal antibody or a fragment thereof according to claim 1.

8. Truncated Midkine protein and a homologues recognized specifically by a monoclonal antibody or a fragment thereof according to claim 1.

9. A monoclonal antibody or a fragment thereof against truncated Midkine protein produced by a hybridoma deposited under accession number FERM P-18069.

10. A monoclonal antibody or a fragment thereof according to claim 9, wherein the truncated Midkine protein has the amino acid sequence composed of full-length amino acid sequence of Midkine protein minus the amino acid sequence encoded in the third exon.

11. A method of detecting truncated Midkine protein comprising detecting truncated Midkine protein expressed specifically in a tumor cell by using a monoclonal antibody or a fragment thereof according to claim 9.

12. A method of detecting a tumor cell comprising detecting truncated Midkine protein especially expressed in a tumor cell by using a monoclonal antibody or a fragment thereof according to claim 9.

13. A kit for detecting truncated Midkine protein, comprising a monoclonal antibody or a fragment thereof according to claim 9.

14. Truncated Midkine protein and a homologues recognized specifically by a monoclonal antibody or a fragment thereof according to claim 9.

15. Hybridoma deposited under accession number FERM P-180069.


SEQUENCE LISTING (8 sequences)

SEQ ID NO: 1 - DNA, 30 bp, Artificial Sequence
  Synthetic DNA
gcccatggggatgaaaaagaaagccgactg

SEQ ID NO: 2 - DNA, 30 bp, Artificial Sequence
  Synthetic DNA
cccaagcttagtcctttcccttccctttct

SEQ ID NO: 3 - DNA, 198 bp, Artificial Sequence
  Synthetic DNA
atgaaaaagaaagccgactgcaagtacaagtttgagaactggggtgcgtgtcatgggggcacaggcaccaaagtccgccaaggcaccctgaagaaggcgcgctacaatgctcagtgccaggagtccatccgcgtcaccaagccctgcacccccaagaccaaagcaaaggccaaagccaagaaagggaagggaaaggac

SEQ ID NO: 4 - protein, 22 aa, Artificial Sequence
  Synthetic Peptide
MKKKADCKYKFENWGACHGGTG

SEQ ID NO: 5 - DNA, 29 bp, Artificial Sequence
  Synthetic DNA
cggaattcggtgcagctgcagcagtctgg

SEQ ID NO: 6 - DNA, 29 bp, Artificial Sequence
  Synthetic DNA
cggctcgagtgaggagacggtgactgagg

SEQ ID NO: 7 - DNA, 27 bp, Artificial Sequence
  Synthetic DNA
gcggatcctgatgttttgatgacccaa

SEQ ID NO: 8 - DNA, 32 bp, Artificial Sequence
  Synthetic DNA
cccaagcttttccaatttggtgcccgctccgg